(19) United States
(12) Patent Application Publication
(10) Pub. No.: US 2011/0250629 A1
(43) Pub. Date: Oct. 13, 2011

(54) ALCOHOL PRODUCTION PROCESS

(75) Inventor: Bjorn Daniel Heijstra, Auckland (NZ)

(73) Assignee: Lanza Tech New Zealand Limited, Parnell (NZ)

(21) Appl. No.: 13/060,357

(22) PCT Filed: Dec. 23, 2010
(86) PCT No.: PCT/NZ10/00266

§ 371 Date: Jun. 29, 2011

Related U.S. Application Data
(60) Provisional application No. 61/289,583, filed on Dec. 23, 2009.

(51) Int. Cl. C12Q 1/02; C12P 7/02; C12P 7/06; C12M 1/36

(52) U.S. Cl. 435/29; 435/286.1; 435/155; 435/161

(57) ABSTRACT

The invention relates to the microbial fermentation of gaseous substrates, particularly to methods of mitigating and/or reducing alcohol toxicity effects on a microbial culture at elevated alcohol concentrations during fermentation. The invention relates particularly to microbial fermentation of substrates comprising CO and the effects of alcohol toxicity are reduced or mitigated by maintaining the temperature below the optimum operating temperature by cooling the fermentation broth.

### FIELD OF THE INVENTION

This invention relates generally to methods for producing products, particularly alcohols, by microbial fermentation. In particular, the invention relates to methods for reducing the effects of alcohol toxicity during the fermentation of substrates comprising CO.

### BACKGROUND OF THE INVENTION

Ethanol is rapidly becoming a major hydrogen-rich liquid transport fuel around the world. Worldwide consumption of ethanol in 2005 was an estimated 12.2 billion gallons. The global market for the fuel ethanol industry has also been predicted to continue to grow sharply in future, due to an increased interest in ethanol in Europe, Japan, the USA and several developing nations.

For example, in the USA, ethanol is used to produce E10, a 10% mixture of ethanol in gasoline. In E10 blends, the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, and as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, and the cultivation of starch or sucrose-producing crops for ethanol production is not economically sustainable in all geographies. Therefore, it is of interest to develop technologies to convert lower cost and/or more abundant carbon resources into fuel ethanol.

CO is a major, free, energy-rich by-product of the incomplete combustion of organic materials such as coal or oil and oil derived products. For example, the steel industry in Australia is reported to produce and release into the atmosphere over 500,000 tonnes of CO annually.

Catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source, all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce ethanol from gases (Abrini et al., Archives of Microbiology 161, pp 345-351 (1994)).

However, ethanol production by micro-organisms by fermentation of gases is always associated with co-production of acetate and/or acetic acid. As some of the available carbon is converted into acetate/acetic acid rather than ethanol, the efficiency of production of ethanol using such fermentation processes may be less than desirable. Also, unless the acetate/acetic acid by-product can be used for some other purpose, it may pose a waste disposal problem. Acetate/acetic acid is converted to methane by micro-organisms and therefore has the potential to contribute to GHG emissions.

Several enzymes known to be associated with the ability of micro-organisms to use carbon monoxide as their sole source of carbon and energy are known to require metal co-factors for their activity. Examples of key enzymes requiring metal cofactor binding for activity include carbon monoxide dehydrogenase (CODH), and acetyl-CoA synthase (ACS).

WO2007/117157, WO2008/115080, WO2009/022925, WO2009/058028, WO2009/064200, WO2009/064201 and WO2009/113878, the disclosure of which are incorporated herein by reference, describe processes that produce alcohols, particularly ethanol, by anaerobic fermentation of gases containing carbon monoxide. Acetate produced as a by-product of the fermentation process described in WO2007/117157 is converted into hydrogen gas and carbon dioxide gas, either or both of which may be used in the anaerobic fermentation process. WO2009/022925 discloses the effect of pH and ORP in the conversion of substrates comprising CO to products such as acids and alcohols by fermentation. WO2009/058028 describes the use of industrial waste gases for the production of products, such as alcohol, by fermentation. WO2009/064201 discloses carriers for CO and the use of CO in fermentation. WO2009/113878 discloses the conversion of acid(s) to alcohol(s) during fermentation of a substrate comprising CO.

In fermentation of substrates comprising CO, where solvents such as alcohol are produced as the major or only product, solvents can accumulate in a fermentation vessel unless they can be effectively removed. It is well established that metabolism and microbial growth slow and eventually stop at elevated fermentation broth alcohol levels due to alcohol toxicity. For example, Tomas et al. state that “accumulation of organic solvents has been shown to permeabilize the cell membrane, resulting in a positive flux of ATP, protons, ions, and macromolecules such as RNA and proteins. Flux of ions dissipates the proton motive force and affects the proton gradient (ΔpH) and electrochemical potential (Δψ), thereby diminishing the energy status of the cell.” Applied and Environmental Microbiology, 2003, 69, 4951-4965. Thus solvents and particularly alcohols have a detrimental effect on microbial fermentation at elevated concentrations. Attempts to alleviate or mitigate this toxicity effect have included time consuming selection procedures, effectively selecting solvent tolerant microbes (such as Williams et al. Appl. Microbiol. Biotechnol. 2007, 74, 422-432 and references therein) and genetic engineering (such as Tomas et al and references therein).

This low natural tolerance of bacteria to alcohols, sets a physical limit for alcohol production if the alcohol is not continuously removed. However, the recovery of alcohol is relatively more energy intensive at lower fermentation broth alcohol levels.

It is an object of the present invention to provide a process that goes at least some way towards overcoming the above disadvantages, or at least to provide the public with a useful choice.

### SUMMARY OF THE INVENTION

In a first broad aspect of the invention, there is provided a method of mitigating and/or reducing alcohol toxicity effects on a microbial culture at elevated alcohol concentrations during fermentation. In particular embodiments, the fermentation is microbial fermentation of a substrate comprising CO to produce products including one or more alcohols. In particular embodiments, one alcohol is ethanol.

Typically, fermentation of substrates comprising CO is performed at or around an optimum operating temperature. However, in accordance with the invention, the method includes maintaining fermentation temperature below the optimum operating temperature, such that the effects of alcohol toxicity are reduced and/or mitigated.

Fermentation of substrates comprising CO are typically conducted in bioreactors, wherein one or more micro-organisms are suspended in a fermentation broth comprising a liquid nutrient medium comprising nutrients essential for microbial growth and/or metabolite production. In particular embodiments, the fermentation temperature can be decreased and/or maintained below the optimum operating temperature by cooling the fermentation broth.

In particular embodiments, the effects of alcohol toxicity are reduced or mitigated by maintaining the temperature of the fermentation broth by up to 1° C. below the optimum operating temperature, or up to 2° C. below the optimum operating temperature, or up to 3° C. below the optimum operating temperature, or up to 4° C. below the optimum operating temperature, or up to 5° C. below the optimum operating temperature, or up to 6° C. below the optimum operating temperature, or up to 7° C. below the optimum operating temperature, or up to 8° C. below the optimum operating temperature, or at least 8° C. below the optimum operating temperature.

In particular embodiments, the effects of alcohol toxicity are reduced and/or mitigated at elevated alcohol levels, wherein the concentration of alcohol in the fermentation broth exceeds 30 g/L, or 35 g/L; or 40 g/L, or 45 g/L; or 50 g/L, or 55 g/L; or 60 g/L, or 65 g/L; or 70 g/L, or 75 g/L; or 80 g/L.

In a second broad aspect, the invention provides a method of regulating fermentation temperature in response to alcohol concentration in a fermentation broth to reduce and/or mitigate the effects of alcohol toxicity. In particular embodiments, the method includes the step of fermenting a substrate comprising CO to produce products including one or more alcohols. In particular embodiments, one alcohol is ethanol.

In particular embodiments, the fermentation temperature can be decreased as alcohol increases above a predetermined threshold.

In another broad aspect, there is provided a method of reducing alcohol toxicity effects on a microbial culture in an alcohol production fermentation, the method including:
a) identifying an optimum operating temperature or range of the microbial culture
b) maintaining the microbial culture at a temperature lower than the optimum operating temperature.

In another aspect, there is provided a method of increasing alcohol concentration in a fermentation broth above a predetermined threshold concentration, the method including:
a) maintaining a fermentation operating temperature at or about an optimum operating temperature of a microbial culture in a fermentation broth, such that the microbial culture converts a substrate to one or more products including one or more alcohols until the concentration of alcohol in the fermentation exceeds the predetermined threshold concentration; then
b) maintaining the fermentation operating temperature below the optimum operating temperature of the microbial culture.

In another broad aspect, the invention provides a microbial fermentation system configured to, in use, reduce alcohol toxicity effects on a microbial culture, the system including:
a) a bioreactor, configured to, in use, contain a fermentation broth;
b) alcohol determining means; configured to, in use, determine alcohol concentration of a fermentation broth;
c) temperature regulating means; configured to, in use, regulate temperature of the fermentation broth.

In particular embodiments, the fermentation temperature is decreased by up to 1° C.; or up to 2° C.; or up to 3° C.; or up to 4° C.; or up to 5° C.; or up to 6° C.; or up to 7° C.; or up to 8° C.; or at least 8° C.

In particular embodiments, the predetermined threshold alcohol concentration is 30 g/L, or 35 g/L; or 40 g/L, or 45 g/L; or 50 g/L, or 55 g/L; or 60 g/L, or 65 g/L; or 70 g/L, or 75 g/L; or 80 g/L.

Those skilled in the art will appreciate suitable means for cooling the fermentation broth. Furthermore, those skilled in the art will appreciate methods for determining alcohol concentration in a fermentation broth at discrete time points or continuously.

In particular embodiments, the alcohol concentration can be determined using Liquid Chromatography, Gas Chromatography, IR, NIR, Mass Spectrometry or combinations thereof.

Embodiments of the invention find particular application in the production of acids and alcohols, particularly ethanol by fermentation of a gaseous substrate comprising CO. The substrate may comprise a gas obtained as a by-product of an industrial process. In certain embodiments, the industrial process is selected from the group consisting of ferrous metal products manufacturing, non-ferrous products manufacturing, petroleum refining processes, gasification of biomass, gasification of coal, electric power production, carbon black production, ammonia production, methanol production and coke manufacturing. In one embodiment of the invention, the gaseous substrate is syngas. In one embodiment, the gaseous substrate comprises a gas obtained from a steel mill.

In particular embodiments of the first and second aspects, the CO-containing substrate will typically contain a major proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

In various embodiments, the fermentation is carried out using a culture of one or more strains of carboxydotrophic bacteria. In various embodiments, the carboxydotrophic bacterium is selected from Clostridium, Moorella, Oxobacter, Peptostreptococcus, Acetobacterium, Eubacterium or Butyribacterium. In one embodiment, the carboxydotrophic bacterium is Clostridium autoethanogenum.

In a particular embodiment, the effects of alcohol toxicity can be reduced or mitigated by adjusting temperature of a microbial culture comprising Clostridium autoethanogenum to below 37° C. In particular embodiments, the temperature of the microbial culture is adjusted to less than 36° C.; or less than 35° C.; or less than 34° C.; or less than 33° C.; or less than 31° C.; or less than 29° C.; or less than 27° C.; or less than 25° C.

In a third broad aspect, the invention provides a system including a bioreactor for fermentation of a substrate comprising CO, means for determining alcohol concentration of a fermentation broth in the bioreactor and means for regulating temperature of the fermentation broth. In particular embodiments, the means for determining and the means for regulating are linked by controlling means such that temperature can be regulated in response to changes in alcohol concentration in accordance with the methods of the first and second aspects.

The invention may also includes the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

The invention will now be described in detail with reference to the accompanying Figures in which:

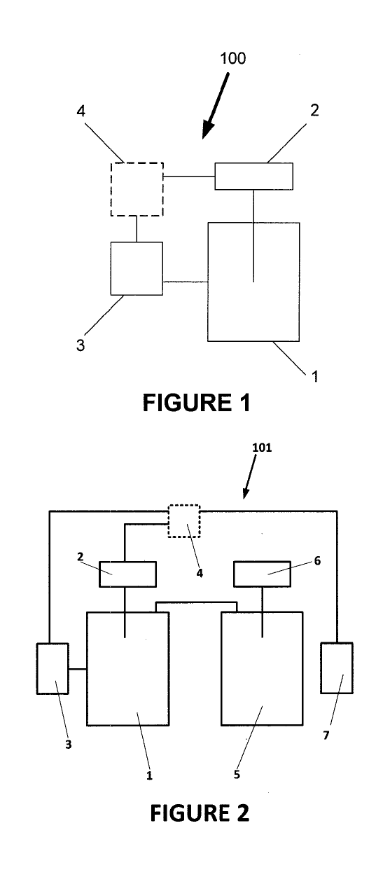

FIG. 1: is a schematic representation according to one embodiment of the invention.

FIG. 2: is a schematic representation according to one embodiment of the invention.

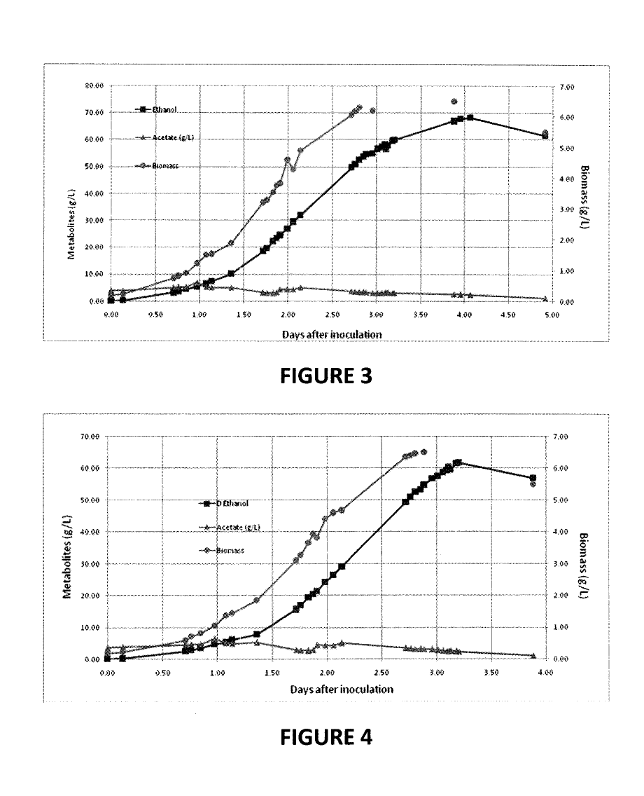

FIG. 3: shows microbial growth and metabolite production as described in example 1 (CSTR A).

FIG. 4: shows microbial growth and metabolite production as described in example 1 (CSTR B).

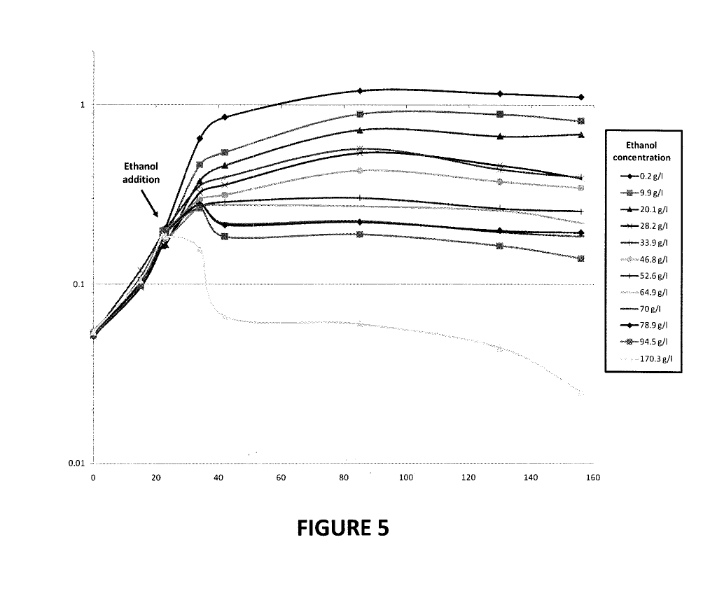

FIG. 5: shows microbial growth and metabolite production as described in example 2.

FIG. 5: shows microbial growth of Clostridium autoethanogenum as described in example 2.

### DETAILED DESCRIPTION OF THE INVENTION

In accordance with the invention, there is provided a method of increasing alcohol production in fermentation. Typically, during fermentation of substrate comprising CO, products, such as acids and/or alcohols are produced. However, in embodiments where alcohol is produced as the major or only product, alcohol can accumulate unless it can be effectively removed. It is well established that metabolism and microbial growth slow down and eventually stop at elevated fermentation broth alcohol level due to alcohol toxicity. For example, Clostridia such as Clostridium autoethanogenum produce products including ethanol. However, at concentrations of over 20-40 g/L, growth rate is partially inhibited and productivity decreases. At concentrations over 40-50 g/L, metabolism is substantially slowed and the growth rate of the microbial culture is also substantially slowed. In order to maintain microbial growth and achieve efficient alcohol production, alcohol levels must be maintained at levels below approximately 40-50 g/L.

It is appreciated that alcohol toxicity levels vary for different micro-organisms. For example ethanol concentrations above 1% are inhibitory to growth of Clostridium thermocellum. However, through strain selection, the micro-organism can still thrive in ethanol concentrations up to 8%.

In accordance with the methods of the invention, a microbial culture can continue to grow and produce products including alcohol even when alcohol has accumulated to elevated levels in the fermentation broth. In particular embodiments of the invention, the fermentation broth is cooled, or allowed to cool such that the microbial culture continues to grow and/or produce products when the broth alcohol concentration is elevated. In accordance with the invention, microbial growth and/or alcohol production is maintained when the alcohol levels in the fermentation broth are at least 30 g/L, or at least 35 g/L, or at least 40 g/L, or at least 45 g/L, or at least 50 g/L, or at least 55 g/L, or at least 60 g/L, or at least 65 g/L, or at least 70 g/L, or at least 75 g/L, or at least 80 g/L.

Without wishing to be bound by theory, it is considered that cooling the fermentation broth reduces or mitigates the toxicity effects of elevated alcohol concentrations. It is further considered that increasing alcohol concentrations in a fermentation broth results in increasing fluidity of the membrane of a microbial cell. Subsequently, at elevated levels of alcohol, the cell membrane becomes less effective. For example, as fluidity increases, the membrane becomes less effective at keeping out potentially toxic compounds, such as free acids, and maintaining the biological gradients and motive forces necessary to sustain viability. As broth alcohol concentrations increase, growth is inhibited and productivity drops. However, as the alcohol concentration increases above a threshold toxic concentration or a concentration range, the fluidity increases above functional levels and the microbial growth will cease and in extreme circumstances the microbial cell will lyse. Thus overall, while elevated alcohol has a softening effect in the cell membrane, effectively decreasing rigidity, decreasing the temperature of the fermentation broth can reduce the softening such that normal cellular functions can continue.

It is appreciated the fluidity of the membrane and the point at which a membrane fails may be dependent on additional factors, such as pH, broth ORP, fermentation nutrient concentrations and/or substrate concentrations. Furthermore, the structure of the microbial membrane is dynamic and can change depending on the extracellular conditions and/or the state of the micro-organism. As such, the microbial membrane may become less effective (more likely to leak undesirable components) over a wide range of elevated alcohol concentrations.

It is hypothesised that micro-organisms can regulate the structure of the membrane over time in response to environmental changes, such as extracellular alcohol concentration. As such, the fluidity and/or rigidity of the membrane can be altered in response to changes in fermentation conditions, such as high alcohol. However, in accordance with the invention, the fluidity of the membrane can be changed quickly, by changing the temperature of the fermentation. Thus, membrane characteristics can be quickly changed such that deleterious effects of increasing alcohol concentrations are avoided or minimised or reversed.

In accordance with the invention, the effects of elevated alcohol in the fermentation broth can be partially or substantially overcome by cooling the microbial culture. It is considered that cooling a microbial cell decreases the fluidity of the membrane, thus mitigating some or all of the deleterious effects of alcohol toxicity. In accordance with the invention, alcohol toxicity at elevated alcohol concentrations is alleviated by decreasing the temperature of the fermentation by up to 1° C., or up to 2° C., or up to 3° C., or up to 4° C., or up to 5° C., or up to 6° C., or up to 7° C., or up to 8° C., or at least 8° C.

### DEFINITIONS

Unless otherwise defined, the following terms as used throughout this specification are defined as follows:

The term “substrate comprising carbon monoxide” and like terms should be understood to include any substrate in which carbon monoxide is available to one or more strains of bacteria for growth and/or fermentation, for example.

“Gaseous substrate comprising carbon monoxide” include any gas which contains carbon monoxide. The gaseous substrate will typically contain a significant proportion of CO, preferably at least about 5% to about 100% CO by volume.

In the context of fermentation products, the term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. The term “acetate” includes both acetate salt alone and a mixture of molecular or free acetic acid and acetate salt, such as the mixture of acetate salt and free acetic acid present in a fermentation broth as may be described herein. The ratio of molecular acetic acid to acetate in the fermentation broth is dependent upon the pH of the system.

The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Moving Bed Biofilm Reactor (MBBR), Bubble Column, Gas Lift Fermenter, Membrane Reactor such as Hollow Fibre Membrane Bioreactor (HFMBR), Static Mixer, or other vessel or other device suitable for gas-liquid contact.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

“Fermentation broth” is the typically aqueous liquid media comprising one or more microorganisms and dissolved nutrients such as metal and mineral salts required for a fermentation.

While the following description focuses on particular embodiments of the invention, namely the production of ethanol and/or acetate using CO as the primary substrate, it should be appreciated that the invention may be applicable to production of alternative alcohols and/or acids and the use of alternative substrates as will be known by persons of ordinary skill in the art to which the invention relates. For example, gaseous substrates containing carbon dioxide and hydrogen may be used. Further, the invention may be applicable to fermentation to produce butyrate, propionate, caproate, ethanol, propanol, and butanol. The methods may also be of use in producing hydrogen. By way of example, these products may be produced by fermentation using microbes from the genus Moorella, Clostridia, Ruminococcus, Acetobacterium, Eubacterium, Butyribacterium, Oxobacter, Methanosarcina, Methanosarcina, and Desulfotomaculum.

### Fermentation

Certain embodiments of the invention are adapted to use gas streams produced by one or more industrial processes. Such processes include steel making processes, particularly processes which produce a gas stream having a high CO content or a CO content above a predetermined level (i.e., 5%). According to such embodiments, acetogenic bacteria are preferably used to produce acids and/or alcohols, particularly ethanol or butanol, within one or more bioreactors. Those skilled in the art will be aware upon consideration of the instant disclosure that the invention may be applied to various industries or waste gas streams, including those of vehicles with an internal combustion engine. Also, those skilled in the art will be aware upon consideration of the instant disclosure that the invention may be applied to other fermentation reactions including those using the same or different micro-organisms. It is therefore intended that the scope of the invention is not limited to the particular embodiments and/or applications described but is instead to be understood in a broader sense; for example, the source of the gas stream is not limiting, other than that at least a component thereof is usable to feed a fermentation reaction. The invention has particular applicability to improving the overall carbon capture and/or production of ethanol and other alcohols from gaseous substrates comprising CO.

Processes for the production of ethanol and other alcohols from gaseous substrates are known. Exemplary processes include those described for example in WO2007/117157, WO2008/115080, WO2009/022925, WO2009/064200, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

A number of anaerobic bacteria are known to be capable of carrying out the fermentation of CO to alcohols, including n-butanol and ethanol, and acetic acid, and are suitable for use in the process of the present invention. Examples of such bacteria that are suitable for use in the invention include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091), Clostridium ragsdalei (WO/2008/028055) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Moorella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp 41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is a Clostridium autoethanogenum having the identifying characteristics of the strain deposited at the German Resource Centre for Biological Material (DSMZ) under the identifying deposit number 19630. In another embodiment, the Clostridium autoethanogenum is a Clostridium autoethanogenum having the identifying characteristics of the strain deposited at the German Resource Centre for Biological Material (DSMZ) under the identifying deposit number 23693. In another embodiment, the Clostridium autoethanogenum is a Clostridium autoethanogenum having the identifying characteristics of DSMZ deposit number DSMZ 10061.

Culturing of the bacteria used in the methods of the invention may be conducted using any number of processes known in the art for culturing and fermenting substrates using anaerobic bacteria. Exemplary techniques are provided in the “Examples” section below. By way of further example, those processes generally described in the following articles using gaseous substrates for fermentation may be utilised: (i) K. T. Klasson, et al. (1991). Bioreactors for synthesis gas fermentations resources. Conservation and Recycling; 5; 145-165; (ii) K. T. Klasson, et al. (1991). Bioreactor design for synthesis gas fermentations. Fuel. 70. 605-614; (iii) K. T. Klasson, et al. (1992). Bioconversion of synthesis gas into liquid or gaseous fuels. Enzyme and Microbial Technology. 14; 602-608; (iv) J. L. Vega, et al. (1989). Study of Gaseous Substrate Fermentation Carbon Monoxide Conversion to Acetate. 2. Continuous Culture. Biotech. Bioeng. 34. 6. 785-793; (v) J. L. Vega, et al. (1989). Study of gaseous substrate fermentations: Carbon monoxide conversion to acetate. 1. Batch culture. Biotechnology and Bioengineering. 34. 6. 774-784; (vi) J. L. Vega, et al. (1990). Design of Bioreactors for Coal Synthesis Gas Fermentations. Resources, Conservation and Recycling. 3. 149-160; all of which are incorporated herein by reference.

The fermentation may be carried out in any suitable bioreactor, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR). Also, in some embodiments of the invention, the bioreactor may comprise a first, growth reactor in which the micro-organisms are cultured, and a second, fermentation reactor, to which fermentation broth from the growth reactor is fed and in which most of the fermentation product (e.g. ethanol and acetate) is produced.

According to various embodiments of the invention, the carbon source for the fermentation reaction is a gaseous substrate containing CO. The substrate may be a CO-containing waste gas obtained as a by-product of an industrial process, or from another source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing substrate may be captured from the industrial process before it is emitted into the atmosphere, using any convenient method. Depending on the composition of the CO-containing substrate, it may also be desirable to treat it to remove any undesired impurities, such as dust particles before introducing it to the fermentation. For example, the gaseous substrate may be filtered or scrubbed using known methods.

Alternatively, the CO-containing substrate may be sourced from the gasification of biomass. The process of gasification involves partial combustion of biomass in a restricted supply of air or oxygen. The resultant gas typically comprises mainly CO and H₂, with minimal volumes of CO₂, methane, ethylene and ethane. For example, biomass by-products obtained during the extraction and processing of foodstuffs such as sugar from sugarcane, or starch from maize or grains, or non-food biomass waste generated by the forestry industry may be gasified to produce a CO-containing gas suitable for use in the present invention.

The CO-containing substrate will typically contain a major proportion of CO, such as at least about 20% to about 100% CO by volume, from 40% to 95% CO by volume, from 40% to 80% CO by volume, from 40% to 70% CO by volume, and from 40% to 60% CO by volume. In particular embodiments, the substrate comprises approximately 25%, or 30%, or 35%, or 40%, or 45%, or 50% CO by volume. Substrates having lower concentrations of CO, such as 6%, may also be appropriate, particularly when H₂ and CO₂ are also present.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H2:CO. In other embodiments, the substrate stream comprises low concentrations of H2, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume.

Typically, the carbon monoxide will be added to the fermentation reaction in a gaseous state. However, the methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October, 2002) could be used for this purpose.

It will be appreciated that for growth of the bacteria and CO-to-alcohol fermentation to occur, in addition to the CO-containing substrate gas, a suitable liquid nutrient medium will need to be fed to the bioreactor. A nutrient medium will contain vitamins and minerals sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438, WO2007/117157, WO2008/115080, WO2009/022925, WO2009/058028, WO2009/064200, WO2009/064201 and WO2009/113878, referred to above. The present invention provides a novel media which has increased efficacy in supporting growth of the micro-organisms and/or alcohol production in the fermentation process. This media will be described in more detail hereinafter.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO07/117,157, WO08/115,080 and WO2009/022925.

The optimum reaction conditions will depend partly on the particular micro-organism used. However, in general, it is preferred that the fermentation be performed at pressure higher than ambient pressure. Operating at increased pressures may allow a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

The benefits of conducting a gas-to-ethanol fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the ethanol product is consumed by the culture.

### Product Recovery

The products of the fermentation reaction can be recovered using known methods. Exemplary methods include those described in WO07/117,157, WO08/115,080, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example only ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which is produced as a by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol- and acetate-containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used in the processes of the present invention. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### Reducing the Effects of Alcohol Toxicity

In accordance with the invention, there is provided a method of mitigating or reducing the effects of alcohol toxicity at elevated alcohol concentrations in microbial fermentation processes producing products including alcohols. In a broad aspect, the invention provides a method of mitigating or reducing the effects of alcohol toxicity in microbial fermentation of a substrate comprising CO. The method includes maintaining the temperature of one or more micro-organisms below an optimum operating temperature. Alcohol toxicity leads to a slowing of growth and metabolite production and can cause a microbial culture to stop growing and producing metabolites entirely. Under extreme conditions, alcohol toxicity can cause microbial cells to lyse. Typically, fermentation of a substrate comprising CO, by carboxydotrophic micro-organisms, such as one or more acetogenic micro-organism, is conducted at an optimum operating temperature.

Alcohol can be removed from a fermentation broth by a number of different means. For example, alcohol concentrations can be maintained at a desired concentration by operating the fermentation in a continuous manner, wherein at steady state, the alcohol concentration in the broth remains substantially constant. The actual concentration will be a function of a number of factors including dilution rate, substrate feed rate, various nutrient concentration levels. Alcohol can also be continuously or semi-continuously removed by other means known to those in the art, such as extractive fermentative techniques, stripping, membrane extraction, all of which may be used in combination with the instant invention.

Fermentation of substrates comprising CO are typically conducted in bioreactors, wherein the micro-organism is suspended in a liquid nutrient media containing nutrients essential for microbial growth and metabolite production. Under such conditions, the culture typically produces acid(s) (such as acetate) and alcohol(s) (such as ethanol). In accordance with the invention, when the fermentation is conducted at temperatures below the optimum operating temperature, the toxicity effects of alcohol are reduced and the microbial culture can continue to grow and produce metabolites even at elevated alcohol levels.

In particular embodiments of the invention, carboxydotrophic bacteria, such as Clostridium autoethanogenum, are cultured at a temperature below the optimum operating temperature, such that the toxicity effects of alcohol are reduced. Typically, carboxydotrophic micro-organisms have optimum operating temperatures in the range 30-70° C. Examples of optimum operating temperature are detailed in “Microbiology of synthesis gas fermentation for biofuels production” A. M. Henstra et al. Current Opinion in Biotechnology, 2007, 18, 200-206. For example, mesophilic bacteria, such as Clostridium autoethanogenum, Clostridium ljungdahli and Clostridium carboxydivorans have an optimum growth and metabolite production temperature of approximately 37° C. However, thermophilic bacteria have significantly higher optimum temperatures of 55-70° C., for example strains of Moorella thermoacetica (55-60° C.), Carboxydothermus hydrogenoformans (70-72° C.), Desulfotomaculum carboxydivorans (60° C.). In particular embodiments, the temperature of the microbial culture is adjusted to more than 1° C. below the optimum operating temperature; or more than 2° C. below the optimum operating temperature; or more than 3° C. below the optimum operating temperature; or more than 4° C. below the optimum operating temperature; or more than 6° C. below the optimum operating temperature; or more than 8° C. below the optimum operating temperature; or more than 10° C. below the optimum operating temperature. In particular embodiments, a microbial culture comprising Clostridium autoethanogenum can be cooled to below 37° C., such that the effects of alcohol toxicity are reduced or mitigated. In particular embodiments, the temperature of the microbial culture is adjusted to less than 36° C.; or less than 35° C.; or less than 34° C.; or less than 33° C.; or less than 31° C.; or less than 29° C.; or less than 27° C.; or less than 25° C.

It is recognised that most microbial fermentations are conducted in a bioreactor, with the microbial culture suspended in a liquid nutrient media. In particular embodiments of the invention, the liquid nutrient media can be cooled, or allowed to cool, such that the effects of alcohol toxicity can be reduced.

Those skilled in the art will appreciate means required to cool a microbial culture will depend on several factors including size and shape of the vessel containing the culture, speed at which the culture is cooled and whether the fermentation is exothermic or endothermic. For example, many large scale fermentation processes need to be externally cooled to remove excess heat generated during the fermentation reaction. The known cooling means already provided may be adapted to further cool the microbial culture in accordance with the methods of the invention. In alternative embodiments, where the microbial culture requires external heating to maintain the optimum operating temperature, the culture may be cooled by removing the heat source and allowing the fermenter to cool to ambient temperature over time. Additionally or alternatively, such cultures may be further cooled using any known refrigeration or cooling means.

In particular embodiments of the invention, the liquid nutrient media is allowed to cool below the optimum operating temperature by removing thermostatic heat control. Under such conditions, the temperature of the liquid nutrient media and the microbial culture will fall to ambient temperature over time. In accordance with the invention, as the temperature of the microbial culture falls below the optimum operating temperature, the effects of alcohol toxicity are reduced.

Under typical fermentation conditions, such as those described herein, a microbial culture can grow and produce metabolites without the deleterious effects (or with minimal effects) of alcohol toxicity at relatively low concentrations of alcohol in a fermentation broth. However, at elevated alcohol concentrations, such as 20-40 g per Litre of fermentation broth, the effects of alcohol toxicity increase. The level or concentration range at which alcohol toxicity starts to deleteriously affect a micro-organism will be dependent on a number of factors, including the micro-organism itself and fermentation parameters such as media conditions. For example, the effects of alcohol toxicity for Clostridium autoethanogenum are observed in the range 20-40 g/L as microbial growth, substrate uptake and metabolite production slow. Similarly, for Clostridium ragsdalei, alcohol toxicity slows down growth and metabolism in the range 20-40 g/L. However, through strain selection techniques described in WO2008/028055 and references therein, the microbial culture can be selected such that the serious effects of alcohol toxicity occur at elevated levels above 50 g/L.

In particular embodiments, the growth rate and the alcohol production rate of a microbial culture is fastest when the broth alcohol concentration is maintained at a low level, such as below 20 g/L, or below 30 g/L, or below 40 g/L. However, in a process, such as the industrial production of alcohol by fermentation, this needs to be balanced against difficulty in recovering low concentrations of alcohol from a fermentation broth. Therefore, in accordance with particular embodiments, the invention provides a first bioreactor maintained at an optimum operating temperature and a second bioreactor maintained below an optimum operating temperature, wherein in use, fermentation broth comprising alcohol and optionally microorganisms pass from the first bioreactor to the second bioreactor, wherein the alcohol concentration can increase. In particular embodiments, the first bioreactor is operated such that the alcohol concentration in the fermentation broth is maintained below 20 g/L, or below 30 g/L, or below 40 g/L, while the alcohol concentration in the second bioreactor can increase to at least 40 g/L, or at least 45 g/L, or at least 50 g/L, or at least 55 g/L, or at least 60 g/L, or at least 65 g/L, or at least 70 g/L.

Other non-CO consuming micro-organisms such as Clostridium thermocellum, are also affected by elevated levels of alcohol in excess of 10 g/L. Again, strains of Clostridium thermocellum have been successfully selected such that the toxicity effects of alcohol are reduced up to 80 g/L alcohol. Those skilled in the art will appreciate that the effects of alcohol toxicity increase with increasing broth alcohol concentration. As such, the methods of the invention are defined by reducing or mitigating the effects of alcohol toxicity on one or more micro-organisms at elevated alcohol concentrations wherein such toxicity effects would typically be observed.

For example in batch fermentation of Clostridium autoethanogenum, the deleterious effects of alcohol toxicity typically become severe at levels over 40 g/L. At elevated alcohol levels over 40 g/L, microbial growth rate and/or metabolite production rate slows. In particular embodiments of the invention, microbial growth stops before alcohol production, as at least a portion of alcohol can be produced by non-growing solventogenic cells. Thus in typical batch fermentations, products including alcohol can accumulate to levels of 50-60 g/L before growth and metabolite production completely stops. However, in accordance with the invention, micro-organisms in batch fermentation can continue to grow and produce alcohol when the broth alcohol level exceeds 60 g/L. For example, when the temperature of the fermentation broth was decreased from the optimum operating temperature of 37° C. to approximately 34° C., the micro-organisms continued to uptake substrate (CO and H2) and produce metabolites up to a fermentation broth concentration of approximately 70 g/L.

In particular embodiments, the invention provides a method of regulating fermentation temperature in response to changes in alcohol concentration. In particular embodiments, during continuous, semi-continuous, batch or fed-batch fermentation, when alcohol levels exceed a predetermined threshold, the temperature of the fermentation broth can be decreased such that alcohol toxicity effects can be reduced or minimised. In accordance with the invention, an operator can monitor broth alcohol concentrations using standard means known in the art and subsequently regulate the fermentation temperature in response to accumulation of elevated alcohol levels. Additionally or alternatively, the concentration of alcohol in a fermentation broth can be monitored automatically, continuously or at discrete time points, and the temperature can be automatically adjusted if the alcohol concentration exceeds a pre-determined set-point or deviates from a predetermined range. It is appreciated automatic control would require some controlling means adapted to monitor alcohol concentration and control temperature regulation means.

Those skilled in the art will appreciate means for manual or automatic determination of alcohol concentrations in a fermentation broth. However, by way of non-limiting example, Liquid Chromatography, Gas Chromatography, IR, NIR, Mass Spectrometry or combinations thereof can be used to determine the concentration of alcohol in a fermentation broth.

In another embodiment of the invention, there is provided a system including a bioreactor for fermentation of a substrate comprising CO, means for determining alcohol concentration of a fermentation broth in the bioreactor and means for regulating temperature of the fermentation broth. In particular embodiments, the means for determining and means for regulating are linked by controlling means such that temperature can be regulated in response to changes in alcohol concentration in accordance with the methods of the invention.

Embodiments of the invention are described by way of example. However, it should be appreciated that particular steps or stages necessary in one embodiment may not be necessary in another. Conversely, steps or stages included in the description of a particular embodiment can be optionally advantageously utilised in embodiments where they are not specifically mentioned.

FIG. 1 is a schematic representation of a system 100, according to one embodiment of the invention. Bioreactor 1 is configured to perform fermentation of substrates, such as substrates comprising CO, to produce products such as alcohol. The fermentation is typically conducted in a liquid nutrient media wherein the substrate can be continuously provided to a microbial culture suspended or immobilised therein. Alcohol concentration in the fermentation broth can be determined using determining means 2. Alcohol concentration can be determined continuously or at discrete time points. Temperature regulating means 3 can be used to regulate the temperature in response to changes in alcohol concentration. Alcohol determining means 2 and temperature regulating means 3 can be linked via optional controlling means 4, which may be optionally linked to a processor (not shown) such that temperature can be automatically regulated in response to changes in alcohol concentration.

FIG. 2 is a schematic representation of a system 101, according to another aspect of the invention, wherein first bioreactor 1 is configured to perform fermentation of substrates, such as substrates comprising CO, to produce products including alcohols, wherein the bioreactor 1 to be operated at or about a predetermined operating temperature and alcohol can be maintained below a predetermined threshold concentration. The optimum operating temperature is maintained or regulated by temperature regulating means 3. In particular embodiments, the first bioreactor 1 is operated as a growth reactor such that microbial growth is promoted. In particular embodiments, the predetermined operating temperature of the first bioreactor 1 is approximately the optimum operating temperature of the microorganism, such as approximately 37 C. In particular embodiments, the predetermined alcohol threshold concentration is less than 20 g/L, or less than 25 g/L, or less than 30 g/L, or less than 35 g/L, or less than 40 g/L of the fermentation broth.

The second bioreactor 5 is configured for the accumulation of alcohol in the fermentation broth and is configured such that alcohol can accumulate above a predetermined threshold value. In particular embodiments, the predetermined threshold concentration in the second bioreactor 5 is the same as the first 1, in other embodiments, the threshold concentration is different. In particular embodiments, the threshold concentration is at least 40 g/L, or at least 45 g/L, or at least 50 g/L, or at least 55 g/L, or at least 60 g/L, or at least 65 g/L, or at least 70 g/L. Thus in accordance with the invention, the temperature regulating means 7 can be sued to maintain the temperature of the fermentation broth in the second bioreactor 5 below the optimum operating temperature, such that in use, the microorganisms can tolerate elevated broth alcohol levels. In particular embodiments, the temperature regulating means 7 is configured to maintain the temperature of a fermentation broth at approx 1° C., or approx 2° C., or approx 3° C., or approx 4° C., or approx 5° C. below the optimum operating temperature of the microorganism.

### EXAMPLES

### Materials and Methods

Bacteria: Clostridium autoethanogenum used is that deposited at the German Resource Centre for Biological Material (DSMZ) and allocated the accession number DSMZ 19630 or DSMZ23693.

### Sampling and Analytical Procedures

Media samples were taken from the CSTR reactor at intervals over periods up to 20 days. Each time the media was sampled care was taken to ensure that no gas was allowed to enter into or escape from the reactor.

### HPLC:

HPLC System Agilent 1100 Series. Mobile Phase: 0.0025N Sulfuric Acid. Flow and pressure: 0.800 mL/min. Column: Alltech IOA; Catalog #9648, 150×6.5 mm, particle size 5 p.m. Temperature of column: 60° C. Detector: Refractive Index. Temperature of detector: 45° C.

### Method for Sample Preparation:

400 μL of sample and 50 μL of 0.15M ZnSO₄ and 50 μL of 0.15M Ba(OH)₂ are loaded into an Eppendorf tube. The tubes are centrifuged for 10 min. at 12,000 rpm, 4° C. 200 μL of the supernatant are transferred into an HPLC vial, and 5 μL are injected into the HPLC instrument.

### Headspace Analysis:

Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s.

### Example 1

### Batch Fermentation in CSTR

Solution A

| | | | | | |
|---|---|---|---|---|---|
| NH₄Ac | 3.083 | g | KCl | 0.15 | g |
| MgC1₂•6H₂O | 0.61 | g | NaCl | 0.12 | g |

| | | | | |
|---|---|---|---|---|
| CaCl₂•2H₂O | 0.294 | g | Distilled Water | Up to 1 L |

Solution(s) B

| Component | mol/L H2O | Component | mol/L H2O |
|---|---|---|---|
| FeCl₃ | 0.1 | Na₂MoO₄ | 0.01 |
| CoCl₂ | 0.05 | ZnCl₂ | 0.01 |
| NiCl₂ | 0.05 | MnCl2 | 0.01 |
| H₃BO₃ | 0.01 | Nitrilotrifluoroacetic acid | 0.3 |
| Na₂SeO₃ | 0.01 | | |

Solution C

| Biotin | 20.0 | mg | Calcium D-(*)- | 50.0 | mg |
|---|---|---|---|---|---|
| Folic acid | 20.0 | mg | pantothenate | | |
| Pyridoxine•HCl | 10.0 | mg | Vitamin B12 | 50.0 | mg |
| Thiamine•HCl | 50.0 | mg | p-Aminobenzoic acid | 50.0 | mg |
| Riboflavin | 50.0 | mg | Thioctic acid | 50.0 | mg |

| | | | | |
|---|---|---|---|---|
| Nicotinic acid | 50.0 | mg | Distilled water | To 1 Litre |

### Preparation of Cr (II) Solution

A 1 L three necked flask was fitted with a gas tight inlet and outlet to allow working under inert gas and subsequent transfer of the desired product into a suitable storage flask. The flask was charged with CrCl₃.6H₂O (40 g, 0.15 mol), zinc granules [20 mesh] (18.3 g, 0.28 mol), mercury (13.55 g, 1 mL, 0.0676 mol) and 500 mL of distilled water. Following flushing with N₂ for one hour, the mixture was warmed to about 80° C. to initiate the reaction. Following two hours of stirring under a constant N₂ flow, the mixture was cooled to room temperature and continuously stirred for another 48 hours by which time the reaction mixture had turned to a deep blue solution. The solution was transferred into N₂ purged serum bottles and stored in the fridge for future use.

Two 2 L CSTR's (A) and (B) were set up under the following conditions: Media was prepared as follows: 85% H₃PO₄ (30 mM) was added to 1.5 L of solution A. The pH of the media was adjusted to 5.3 by the addition of NH4OH. The media solution was sterilised by autoclaving for 30 minutes at 121° C., or by filter sterilisation prior to use. Resazurin was added as a redox indicator. The media solution was aseptically and anaerobically transferred into a 1.5 L CSTR vessel, and continuously sparged with N₂.

Once transferred to the fermentation vessel, the reduction state and pH of the transferred media could be measured directly via probes. The media was heated to 37° C. and stirred at 300 rpm.

Sodium sulfide solution (3.75 mL of a 0.2M solution) was added, followed by trace metal solution B (1.5 mL), Na2WO4 (1.5 mL of a 0.01M solution) then Solution C (15 mL). ORP of the solution was adjusted to approx −200 mV using Cr(II) solution.

Prior to inoculation, the gas in one CSTR (A) was switched to a blend of 33% H2, 23% N2, 31% CO₃ 13% CO2, while the other (B) was switched to a blend of 45% H2, 19% N2, 26% CO, and 10% CO2.

The CSTR's were operated under substantially similar conditions and substrate supply was increased in response to the requirements of each microbial culture.

An actively growing Clostridium autoethanogenum culture (DSMZ19630) was inoculated into the CSTR at a level of approximately 10% (v/v). During this experiment, Na2S solution was added at a rate of approx 0.16 mMol/day.

At day approximately 2.9, the temperature in CSTR (A) was allowed to drop to approximately 34° C., whereas the temperature in CSTR (B) was maintained at 37° C. FIG. 2 shows microbial growth and metabolite production in CSTR (A) and FIG. 3 shows microbial growth and metabolite production in CSTR (B). At day 2.9, the alcohol concentration in CSTR (A) and (B) is approximately the same at approximately 55 g/L. At day 2.9, microbial growth stops in CSTR (B) and metabolite production and substrate uptake (not shown) slow until approximately day 3.4. However, in CSTR (A), microbial growth continues after day 2.9 and Metabolite production and gas uptake (not shown) continue until approximately day 4.2. Continued metabolic activity at elevated alcohol concentrations (CSTR A) allows alcohol to accumulate to approximately 70 g/L in the fermentation broth.

When the temperature of the fermentation is maintained, alcohol toxicity effects quickly prevent further growth, metabolite production and substrate uptake. However, when the temperature of the fermentation broth is reduced, growth, metabolite production and substrate uptake can continue. This shows that reducing temperature reduces or mitigates the effect of alcohol toxicity at elevated alcohol concentrations.

### Example 2

The ethanol tolerance of Clostridium autoethanogenum DSM23693 was tested in serum bottles (FIG. 5). Ethanol was added in various concentrations to an active growing culture at 37° C. in PETC medium (Tab. 1) with 30 psi steel mill gas as substrate (approx 48% CO, 32% N2, 2% H2, and 18% CO2). Ethanol concentrations were confirmed by HPLC analysis using an Agilent 1100 Series HPLC system equipped with a RID operated at 35° C. (Refractive Index Detector) and an Alltech IOA-2000 Organic acid column (150×6.5 mm, particle size 5 μm) kept at 60° C. Slightly acidified water was used (0.005 M H₂SO₄) as mobile phase with a flow rate of 0.7 ml/min. To remove proteins and other cell residues, 400 μl samples were mixed with 100 μl of a 2% (w/v) 5-Sulfosalicylic acid and centrifuged at 14,000×g for 3 min to separate precipitated residues. 10 μl of the supernatant were then injected into the HPLC for analyses.

At the lowest alcohol concentration (0.2 g/L, wherein no additional ethanol was added), the microbial biomass increases from approx 0.2 g/L to approx 0.85 g/L over a 24 hour period. However, at 20 g/L, the microbial biomass increases to only 0.45 g/L over the same period, showing a slowing of growth. Growth was found to be inhibited already at concentrations between 10-20 g/l ethanol, while growth completely ceased after addition of >50 g/l or >5% ethanol.

TABLE 1
PETC medium

| Media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution (see below) | 10 | ml |
| Wolfe's vitamin solution (see below) | 10 | ml |
| Yeast Extract | 1 | g |
| Resazurin (2 g/L stock) | 0.5 | ml |
| NaHCO₃ | 2 | g |
| Reducing agent | 0.006-0.008% (v/v) | |
| Wolfe's vitamin solution | per L of stock | |
| Biotin | 2 | mg |
| Folic acid | 2 | mg |
| Pyridoxine hydrochloride | 10 | mg |
| Thiamine•HCl | 5 | mg |
| Riboflavin | 5 | mg |
| Nicotinic acid | 5 | mg |
| Calcium D-(+)-pantothenate | 5 | mg |
| Vitamin B₁₂ | 0.1 | mg |
| p-Aminobenzoic acid | 5 | mg |
| Thioctic acid | 5 | mg |
| Trace metal solution | per L of Mock | |
| Nitrilotriacetic Acid | 2 | g |
| MnSO₄•H₂O | 1 | g |
| Fe (SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |
| Reducing agent stock | per 100 mL of stock | |
| NaOH | 0.9 | g |
| Cystein•HCl | 4 | g |
| Na₂S | 4 | g |

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications.

Furthermore, titles, heading, or the like are provided to enhance the reader's comprehension of this document, and should not be read as limiting the scope of the present invention. The entire disclosures of all applications, patents and publications cited above and below, if any, are herein incorporated by reference.

The reference to any prior art in this specification is not, and should not be taken as, an acknowledgement or any form of suggestion that that prior art forms part of the common general knowledge in the field of endeavour in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1-20. (canceled)

21. A method of reducing alcohol toxicity effects on a microbial culture in an alcohol production fermentation, said method comprising:
identifying an optimum operating temperature or range of the microbial culture
maintaining the microbial culture at a temperature lower than the optimum operating temperature.

22. The method of claim 21, wherein the fermentation converts a substrate that includes CO into one or more products, said products including one or more alcohols.

23. The method of claim 22, wherein the alcohol is ethanol.

24. The method of claim 21, wherein maintaining the microbial culture comprises maintaining the microbial culture at a temperature at least 1° C. lower than the optimum operating temperature of the microbial culture.

25. The method of claim 21, wherein the microbial culture comprises one or more carboxydotrophic microorganisms.

26. The method of claim 25, wherein the carboxydotrophic microorganisms are alcohol producing acetogens.

27. The method of claim 26, wherein the alcohol producing acetogens are selected from the group consisting of Clostridium autoethanogenum, Clostridium ragsdalei, and Clostridium Ljungdahlii.

28. A method of increasing alcohol concentration in a fermentation broth above a predetermined threshold concentration, said method comprising:
maintaining a fermentation operating temperature at or about an optimum operating temperature of a microbial culture in a fermentation broth, such that the microbial culture converts a substrate to one or more products including one or more alcohols until the concentration of alcohol in the fermentation exceeds the predetermined threshold concentration; then
maintaining the fermentation operating temperature below the optimum operating temperature of the microbial culture.

29. The method of claim 28, wherein the substrate comprises CO.

30. The method of claim 28, wherein the alcohol is ethanol.

31. The method of claim 28, wherein maintaining the fermentation operating temperature below the optimum operating temperature of the microbial culture comprises maintaining the fermentation operating temperature at least 1° C. less than the optimum operating temperature.

32. The method of claim 28, wherein the predetermined threshold concentration is at least 40 grams of ethanol per liter of fermentation broth.

33. The method of claim 28, wherein the microbial culture comprises one or more carboxydotrophic microorganisms.

34. The method of claim 33, wherein the carboxydotrophic microorganisms are alcohol producing acetogens.

35. The method of claim 34, wherein the alcohol producing acetogens are selected from the group consisting of Clostridium autoethanogenum, Clostridium ragsdalei, and Clostridium Ljungdahlii.

36. A microbial fermentation system configured to, in use, reduce alcohol toxicity effects on a microbial culture, said system comprising:
a bioreactor, configured to, in use, contain a fermentation broth;
means for determining alcohol concentration of the fermentation broth;
means for regulating temperature of the fermentation broth.

37. The system of claim 36, wherein the means for regulating temperature and the means for determining alcohol concentration are operatively linked such that, in use, as alcohol concentration increases above a predetermined threshold value in a fermentation broth, the means for regulating temperature reduces the temperature of the fermentation broth.
